(19) United States
(12) Patent Application Publication
(10) Pub. No.: US 2004/0224370 A1
(43) Pub. Date: Nov. 11, 2004

(54) Fluorescence guided cell capture

(76) Inventors: Ping Jiang, San Diego, CA (US); Meng Yang, San Diego, CA (US); Mingxu Xu, La Jolla, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/712,782

(22) Filed: Nov. 12, 2003

Related U.S. Application Data
(60) Provisional application No. 60/425,776, filed on Nov. 12, 2002.

(51) Int. Cl. C12Q 1/68; G01N 33/574; C07K 14/435

(52) U.S. Cl. 435/7.23; 435/325; 530/350

(57) ABSTRACT

Methods of recovering desired living cells contained in a tissue sample are based on separation based on fluorescence of a fluorescent protein produced by the desired cells.

### CROSS-REFERENCE TO RELATED APPLICATIONS

This application claims priority under 35 U.S.C. § 119(e) from provisional application 60/425,776 filed 12 Nov. 2002. The contents of that application are incorporated herein by reference.

### TECHNICAL FIELD

The invention relates to the use of expressed fluorescent proteins as guides for excision of desired tissue, especially tumor tissue. The excised tissue can be tested for metastasis, drug resistance, and expression profile.

### BACKGROUND ART

A variety of fluorescent proteins is known in the art. The first discovered was green fluorescent protein (GFP) from Aequorea victoria; more recently another family of proteins was isolated from coral. Manipulations of the originally isolated and cloned green fluorescent proteins has resulted in the production of proteins whose fluorescence spans a wide range of colors over the entire visible spectrum. These proteins have been used widely as markers in a number of contexts, including whole body imaging of tumor growth, metastasis and angiogenesis (Yang, M., et al., Proc. Natl. Acad. Sci. USA (2002) 99:3824-3829; Yang, M., et al., Proc. Natl. Acad. Sci. USA (2000) 97:1206-1211; Yang, M., et al., Proc. Natl. Acad. Sci. USA (2001) 98:2616-2621). These proteins have also been used in studies of infectious disease (Zhao, M., et al., Proc. Natl. Acad. Sci. USA (2001) 98:9814-9818) and in studies of gene expression (Yang, M., et al., Proc. Natl. Acad. Sci. USA (2000) 12278-12282). A summary of the use of fluorescent proteins in imaging is found in Hoffman, R. M., The Lancet Oncology (2002) 3:546-556.

Although these fluorescent proteins have been widely used as markers as described above, no advantage appears to have been taken of the ability to label cells selectively with these fluorescent proteins in order to provide guidance for excision of desired cell samples. Prior art methods for obtaining precise cell samples has focused on staining with histochemical stains in order to visualize cell locations. The use of such stains results in cell death, and thus the further behavior of the isolated cell samples cannot be examined. A recent embodiment of this technique employs laser capture microdissection wherein a stained section to be microdissected is placed on a microscope slide, the area to be microdigsected is located, and then microdissected with a laser. Typically, this method employs a 20× objective and adjusts the laser spot size to 7.5 μm. If these cells are to analyzed for expression, they are collected and treated with lysis buffer and then processed for RNA extraction. This general procedure is described in Sgroi, D. C., et al., Cancer Res. (1999) 59:5656-5661; Paweletz, C. P., et al., Cancer Res. (2000) 60:6293-6297; Frost, A. R., et al., Current Protocols in Molecular Biology, pp. 25A.21.21-25A.21.24, John Wiley & Sons (2001).

### DISCLOSURE OF THE INVENTION

The present invention provides precise guidance for excision of desired cell samples, as small as a single cell, by using fluorescent protein expression by the desired cell as a guide. As the fluorescent proteins are expressed in living cells, the excised cell sample can be employed in further study for its behavior with respect to, for example, drug resistance. Typically, the cells are contained in a tissue sample; the methods of the invention can also be used on cultured cells. However, a major advantage of the invention methods is that tissue samples of living cells can be employed and the recovered cells with the desired characteristics can be obtained in living form so that further manipulations relevant to these cells can be performed.

Thus, in one aspect, the invention is directed to a method to obtain a desired cell sample which method comprises separating, from within a sample comprising cells, labeled with a first fluorescent protein by virtue of expression and unlabeled cells or cells labeled with a different fluorescent protein, those cells labeled with said first fluorescent protein. The separation may be done using any convenient technique, including microsurgical techniques, fluorescence cell sorting, and the like. The excised cells can then be studied for any desired characteristic as the cells remain alive and possessed of their characteristic properties. An important embodiment employs tumor cells as those selected.

### MODES OF CARRYING OUT THE INVENTION

As used herein, a “fluorescent protein” refers to a protein that, upon appropriate stimulation, will emit light. Typically, a fluorescent protein emits light in the visible range—i.e., in the range of about 400 nm-800 nm when illuminated with an excitation wavelength. When instrumentation is employed, a wide range may be used. Other mechanisms for evoking fluorescence can also be employed—e.g., as in the case of luciferase where metabolic energy effects signal generation. However, proteins that emit visible light upon excitation with higher energy radiation are preferred, especially for in vivo use. The method of the invention is particularly useful in studying tumor tissue due to the ease with which selectivity may be obtained for expression of the fluorescent protein and due to the inherent utility of studying such tissue. Tumor tissue of any origin may be employed, including tumors of the liver, lung, bone, lymph node, breast, ovary, prostate and the like. Metastatic tissue derived from these primary tumors is also of interest.

The label used in the various aspects of the invention is a fluorescent protein. The native gene encoding the seminal protein in this class, green fluorescent protein (GFP) has been cloned from the bioluminescent jellyfish Aequorea victoria (Morin, J., et al., J. Cell Physiol (1972) 77:313-318). The availability of the gene has made it possible to use GFP as a marker for gene expression. The original GFP itself is a 283 amino acid protein with a molecular weight of 27 kD. It requires no additional proteins from its native source nor does it require substrates or cofactors available only in its native source in order to fluoresce. (Prasher, D. C., et al., Gene (1992) 111:229-233; Yang, F., et al., Nature Biotechnol (1996) 14:1252-1256; Cody, C. W., et al., Biochemistry (1993) 32:1212-1218.) Mutants of the original GFP gene have been found useful to enhance expression and to modify excitation and fluorescence, so that “GFP” in various colors, including reds and blues has been obtained. GFP-S65T (wherein serine at 65 is replaced with threonine) is particularly useful in the present invention method and has a single excitation peak at 490 nm. (Heim, R., et al., Nature (1995) 373:663-664); U.S. Pat. No. 5,625,048. Other mutants have also been disclosed by Delagrade, S., et al., Biotechnology (1995) 13:151-154; Cormack, B., et al., Gene (1996) 173:33-38 and Cramer, A., et al., Nature Biotechnol (1996) 14:315-319. Additional mutants are also disclosed in U.S. Pat. No. 5,625,048. By suitable modification, the spectrum of light emitted by the GFP can be altered. Thus, although the term “GFP” is often used in the present application, the proteins included within this definition are not necessarily green in appearance. Various forms of GFP exhibit colors other than green and these, too, are included within the definition of“GFP” and are useful in the methods and materials of the invention. In addition, it is noted that green fluorescent proteins falling within the definition of“GFP” herein have been isolated from other organisms, such as the sea pansy, Renilla reniformis. Any suitable and convenient form of GFP can be used to modify the infectious agents useful in the invention, both native and mutated forms.

In order to avoid confusion, the simple term “fluorescent protein” will be used; in general, this is understood to refer to the fluorescent proteins which are produced by various organisms, such as Renilla and Aequorea as well as modified forms of these native fluorescent proteins which may fluoresce in various visible colors. In general, the terms “fluorescent protein” and “GFP” are used interchangeably.

The cells producing a first fluorescent protein are thus labeled by virtue of this fluorescence; the label is endogenous to the cells and not supplied by external labeling, such as utilization of labeled ligands or antibodies. This allows the cells to metabolize in a normal fashion without disruption from the label. The expression system for the fluorescent protein may be supplied to the cells by techniques generally known in the art, typically by transduction with viral vectors, including retroviral vectors. Methods to obtain cells stably transformed with expression systems for green fluorescent protein (used herein as a generic term for fluorescent proteins in general) are described in U.S. Pat. No. 6,232,523, incorporated herein by reference.

The cells to be modified to produce fluorescent protein can be any type of cells, but are typically animal cells, in particular mammalian cells, avian cells, and the like. Of particular importance are cells that occur in intact tissue, including metastatic tissue.

The methods of transformation will depend on the nature of the cells and would include, for example, lipofection, electroporation, and viral infection, as a none-limiting list. For plant cells, Agrobacterium-mediated transformation can also be used, as well as modification of protoplasts. The choice of control sequences for the expression systems containing the nucleotide sequences encoding the proteins can also be varied and the choice of the appropriate controls and vectors will depend on the nature of the cells and the mode of cell modification.

In order to assure stable modification, including instances where the relevant expression systems may be integrated into the genome, the cells are subjected to selection pressure. Suitable selection markers will depend on the nature of the cells; G418 or hygromycin resistance is a convenient marker for a wide variety of cells; other alternative methods of selection include the use of a toxin such as methotrexate with respect to DHFR based systems. Those of ordinary skill will understand the type of selection to be employed.

The invention provides a method to obtain defined samples of living cells by utilizing selective expression of fluorescent protein to identify cells to be separated from a sample and to discard irrelevant surrounding tissue. The method takes advantage of techniques which provide for selective expression of fluorescent protein in the desired cells. In-one embodiment, the fluorescent protein may be placed under control of a tissue-specific promoter; alternatively, selectivity is obtained through the choice of methods of transfection or cellular modification. For example, with regard to the former, control sequences that are specific for bone, muscle, neural tissue, and the like may be employed. For the latter, retroviral mediated transfection selects for cells that are rapidly dividing, such as tumor cells. Alternatively, the expression system for the fluorescent protein might be directly inserted into the type of tissue to be recovered.

A major application of the method of the invention is, indeed, in the study of tumors. Tumor tissue can be selectively targeted with the relevant expression system as described above. The fluorescent tumor cells can then be mechanically separated from surrounding normal tissue using the fluorescence emission as a guide, or may be separated using cell sorting techniques which rely on fluorescence. As little as a single cell may be isolated in this manner. This is especially important since, if there is some heterogeneity in the cell sample due to incomplete selectivity for expression, because a single cell can be recovered, the heterogeneity can be detected by subsequent behavior of the cells descended from each individual cell. For example, growth patterns from a single isolated tumor-cell will differ from growth patterns exhibited by a non-tumor cell.

In one particularly important application of the technique of the present invention, human tumors can be studied using the recovery technique of the invention. The tumor may be labeled in the subject by utilization of retroviral vectors or by direct administration of the expression system to the tumor. Preferably, however, the tumor sample is first transferred to an immunocompromised experimental animal, such as a rabbit, mouse, rat, or other convenient model. The tumor sample, preferably transplanted orthotopically into the immunocompromised model, is labeled with fluorescent protein using the above-described techniques. The expression of the fluorescent protein in the model system permits identification of suitable tissue for subsequent transplantation; the transplant may be derived from the tumor itself, or from metastases originating from the tumor which will, themselves, be fluorescent. Differences in gene expression in the primary tumor and in its metastases may then be determined by suitable known methods which involve extraction of RNA and subsequent analysis using gene chips or extraction of proteins and analysis by proteomics.

For example, a tumor transplanted from a human subject to an immunocompromised laboratory animal may then be labeled and excised using the label as a guide. The excised tumor can then be itself divided and passaged to a multiplicity of animals, typically a minimum of 50 to provide sufficient subjects for testing of drugs of various types and to study dormancy, metastasis, drug resistance, and the like. Differences may be determined with regard to these parameters depending on the origin of the tumor portion excised—et al., whether primary tumor or metastatic tissue and depending on the location within the primary tumor and the location of the metastasis. At any point in this assessment, a single cell or a defined multiplicity of cells from a specific location may be isolated using the fluorescent protein as a guide and analyzed for gene expression using standard procedures as set forth above.

In one particular embodiment, a patient tumor is orthotopically implanted into an immunocompromised mouse, such as a SCID mouse or nude mouse as described in U.S. Pat. Nos. 5,491,284 and 5,569,812, incorporated herein by reference. The tumor is labeled by modifying it to express a fluorescent protein, e.g., by retroviral transformation. The implanted tumor is allowed to grow and metastasize and portions of the tumor are transplanted orthotopically into an additional 50 immunocompromised mice. Retaining one group of five mice as a control, nine drugs are tested against the tumor in the remaining nine groups of five mice each, assessing the ability of the drug to slow tumor growth. Similar procedures may be performed using metastatic locations as the origin of tumor cells.

Alterations in expression patterns attributable to administration of the drug are detected by excising the tumor cells according to the invention (identified by fluorescence) and extracting RNA for analysis. The RNA may be converted to cDNA, and analyzed using standard gene chip technology.

Alternatively, the tumor cells may be labeled by transforming a tumor cell line with an expression system for a fluorescent protein, culturing the cells thus transformed, injecting the cells into immunocompromised hosts subcutaneously and permitting solid tumors to form. The solid tumors can then be transplanted to the organ of their original origin using surgical orthotopic implantation. These techniques, as well as use of retroviral transformation to label already existing tumors are described in U.S. Pat. Nos. 6,232,523; 6,235,967; 6,235,968; and 6,251,384, all incorporated herein by reference.

In addition, the labels associated with the cells to be separated need not be confined to expression in the desired cell. Thus, the host organism as a whole can be modified to express a fluorescent protein which emits a different wavelength, thus permitting additional contrast with the labeled desired cells.

By using the invention methods, individual cells can be obtained which maintain their living state and which inherently provide a fluorescent label. This is in contrast to standard labeling of cells in tissue culture by utilizing fluorescent labeled antibodies, for example, as would be employed in standard flow cytometry cell sorting techniques. The techniques of the present invention utilize, therefore, endogenous fluorescence provided by the cells themselves as guides for separation, whether done by flow cytometry or by microsurgical techniques.

In addition, the cells to be separated can be dual labeled, wherein the nucleus and cytoplasm emit different colors. In this embodiment, the cells to be modified are transfected with a suitable vector comprising an expression system for each of the fluorescent proteins, one and only one of the fluorescent proteins being coupled to an additional amino acid sequence which will target that protein to the nucleus. The vectors used for transformation may be separate vectors for the fluorescent protein destined for the cytoplasm and the fluorescent protein of a different color destined for the nucleus or both expression systems can be contained on the same expression vector. The nuclear targeting sequence may be employed first, followed by transfection so that the cells contain the expression system for the fluorescent protein that will label the cytoplasm, preferably assuring the stability of the cell line between transfection events in order to assure stability. The order of transfection could also be reversed with the expression system for the cytoplasmic protein administered first. Alternatively, both expression vectors might be contacted with the cell simultaneously, preferably using different selection markers to assure co-transfection. It would also be possible to use a bicistronic expression system for both proteins.

Thus, in one approach, a suitable cell line is infected with a retroviral vector comprising an expression system wherein a nucleotide sequence encoding a fluorescent protein which emits blue light fused to an amino acid sequence encoding a nuclear targeting signal. The viral vector further contains hygromycin resistance as a selectable marker. The treated cells are then subjected to selection pressure in the presence of hygromycin and after several rounds of selection, stable transformants are obtained. The stably transformed cells are then treated with DNA using electroporation wherein the vector comprises green emitting fluorescent protein coupled to DHFR. The cells are subjected then to rounds of selection with both hygromycin and methotrexate to obtain a cell line wherein the nucleus is stained blue and the cytoplasm green.

The differential staining obtainable by the method of the invention is useful in view: of the fact that various portions of the cell cycle give rise to different distributions and/or intensities of radiation emitted from the nucleus and the cytoplasm. Thus, for example, the ratio of intensities will permit determination of cell cycle position. In addition, the morphology of the nucleus is altered when apoptosis occurs and this can readily be detected. The effect-of various agents, including various small molecule drugs, proteins, antisense or triplex forming nucleic acids or inhibitor RNA can be tested by observing the effects of these agents on the cellular cycle or morphology. Differential targeting of various agents to the cytoplasm or to the-nucleus can also be observed using the methods of the invention. The characteristics of the cells that can be evaluated include dormancy, apoptosis, stage of cell cycle, location of targeting agents, and a multiplicity of other characteristics that will familiar to the artisan. If desired, agents used to treat the cells may themselves be labeled.

Thus, if the cells are to observed through a microscope in culture, the agent may be added directly to the culture. If the cells are to observed in a living animal or plant, the agent is typically administered directly to the animal or plant.

For example, dual color PC-3 cells were isolated that express GFP exclusively in the nucleus due to fusion of GFP with histone H2B (21) and express RFP exclusively in the cytoplasm. These cells demonstrate the feasibility of dual color imaging of live prostate cancer cells. PC-3 cells are transformed with pLNC DsRed-2 which is produced from PT67 packaging cells. The DsRed-s expression in the PC-3 cells was monitored under fluorescence microscopy. Selection is with increasing amounts of G418. The DsRed-2 PC-3 cells are transfected with pLHC H2B-GFP DNA using LIPOFECTMINE Plus™. After 24 hours incubation, the H2B GFP and DsRed-2-expressing cells are selected by increasing amounts of both hygromycin and G418.

The cells to be recovered may also be an infectious agent. The nucleotide sequence encoding the fluorescent protein may be introduced into the infectious agent by direct modification, such as modification of a viral genome to locate the fluorescent protein encoding sequence in a suitable position under the control sequences indigenous to the virus, or may be introduced into microbial systems using appropriate expression vectors. Infective agents may be bacteria, eukaryotes such as yeast, protozoans such as malaria, or viruses. A multiplicity of expression vectors for particular types of bacterial, protozoan, and eukaryotic microbial systems is well known in the art. A litany of control sequences operable in these systems is by this time well understood. The infectious agent is thus initially modified either to express the fluorescent protein under control of a constitutive promoter as a constant feature of cell growth and reproduction, or may be placed in the microbial or viral genome at particular desired locations, replacing indigenous sequences which may be involved in virulence or otherwise in the progress of infection to study the temporal and spatial parameters characteristic of expression of these indigenous genes. Thus, it is possible to-explore the types of factors indigenous to the microbe or virus which contribute to the effectiveness of the infection by suitable choice of positioning.

The appropriately modified infectious agent is then administered to the subject in a manner which mimics, if desired, the route of infection believed used by the agent or by an arbitrary route. Administration may be by injection, gavage, oral, by aerosol into the respiratory system, by suppository, by contact with a mucosal surface in general, or by any suitable means known in the art to introduce infectious agents. Unlike the situation with regard to the study of tumor metastasis using fluorescence, it is not necessary that the subject be immunocompromised since infection occurs readily in organisms with intact immune systems. However, immunocompromised subjects may also be useful in studying the progress of the condition.

Although endoscopy can be used as well as excision of individual tissues, it is particularly convenient to visualize the migration of infective agent and infected cells in the intact animal through fluorescent optical tumor imaging (FOTI). This permits real-time observation and monitoring of progression of infection on a continuous basis, in particular, in model systems, in evaluation of potential anti-infective drugs and protocols. Thus, the inhibition of infection observed directly in test animals administered a candidate drug or protocol in comparison to controls which have not been administered the drug or protocol indicates the efficacy of the candidate and its potential as a treatment. In subjects being treated for infection the availability of FOTI permits those devising treatment protocols to be informed on a continuous basis of the advisability of modifying or not modifying the protocol.

The distributed infective cells can then be recovered from the tissues of the subject using the method of the invention, employing microsurgical techniques and/or cell sorting techniques.

In one embodiment, cells whose recovery is desired are labeled with a first fluorescent protein and cells that are not to be recovered are simply unlabeled. Thus, tumor cells or cells derived from a particular organ can be selectively labeled and the remaining cells in the tissue sample are modified. Alternatively, “background” cells contained in the tissue may also be labeled with; a fluorescent protein that emits a wavelength different from that of the first fluorescent protein. These cells can have introduced label using techniques similar to those employed with the desired cells.

1. A method to recover one or more desired cells from a tissue sample, which method comprises separating one or-more living cells, contained in the sample, that produce a first fluorescent protein
from cells contained in the sample that do not produce said first fluorescent protein,
thereby recovering one or more living cells that produce said first fluorescent protein.

2. The method of claim 1, wherein the cells that produce the first fluorescent protein are tumor cells.

3. The method of claim 1, wherein said separating is by surgical procedures.

4. The method of claim 1, wherein said separating is by fluorescent cell sorting.

5. The method of claim 2, wherein the tumor cells are metastatic tumor cells of the lung, bone, lymph node or liver.

6. The method of claim 1, wherein the first fluorescent protein is a green fluorescent protein or a red fluorescent protein.

7. The method of claim 1, wherein said one or more living cells recovered consists of a single living cell.

8. The method of claim 1, wherein said cells that produce said first fluorescent protein are present in an immunocompromised laboratory animal.

9. The method of claim 8, which further comprises identifying said cells that produce the first fluorescent protein by monitoring fluorescence and transferring portions of said cells to additional immunocompromised animals.

10. The method of claim 1, which further comprises subjecting the recovered one or more living cells that produce said first fluorescent protein to gene expression analysis.

11. The method of claim 1, wherein said cells contained in the sample that do not produce the first fluorescent protein produce a second fluorescent protein that emits a different wavelength from the first fluorescent protein.
